SEQUENCE LISTING (38 sequences)

SEQ ID NO: 1 - protein, 349 aa, Chlorella vulgaris
MRNLTQSVSGISQADHQPHSVEAERLKLQYLVNLCAASDAEPQVGSDVIQGLTQTPKTLPPQYFYDDRGSVLFEEICELPEYYPTRTEAAILRQYASEIARMTGACELVELGSGSSTKTRLLLDAYAAHAYPLQYVPIDVSAGILESSARQLLADYPSLQVQGLVGTYELALQQLTPTLLPTRMIVFLGSTLGNFNPQECDVFFSQIQGALKVGEYFLLGVDLQKAKPLLEDAYNDSQGVTAEFNLNMLEHLNQRFAGNFDTQQFEHWAFYNEQLNQIEMHLRCLEAHTVRLEALDLTLNFAAGETIMTEISRKFDLEGIQQELQVKGLKPLKVWTDSQQWFGLILCQR

SEQ ID NO: 2 - DNA, 1050 bp, Chlorella vulgaris
  source 1..1050: mol_type = genomic DNA
atgcgcaacctgacgcagagcgtgagcggcattagccaagcggatcatcagccgcatagcgtggaagcggaacgcctgaaactgcagtatctggtgaacctgtgcgcggcgagcgatgcggaaccgcaagtgggcagcgatgtgattcaaggcctgacgcagaccccgaaaaccctgccgccgcagtatttttatgatgatcgcggcagcgtgctgtttgaagaaatttgcgaactgccggaatattatccgacccgcaccgaagcggcgattctgcgtcagtatgcgagcgaaattgcgcgcatgaccggcgcgtgcgaactggtggaactgggcagcggcagcagcaccaaaacccgcctgctgctggatgcgtatgcggcgcatgcgtatccgctgcagtatgtgccgattgatgtgagcgcgggcattctggaaagcagcgcgcgtcagctgctggcggattatccgagcctgcaagtgcaaggcctggtgggcacctatgaactggcgctgcagcagctgaccccgaccctgctgccgacccgcatgattgtgtttctgggcagcaccctgggcaactttaacccgcaagaatgcgatgtgttttttagtcagattcaaggcgcgctgaaagtgggcgaatattttctgctgggcgtggatctgcagaaagcgaaaccgctgctggaagatgcgtataacgatagccaaggcgtgaccgcggaatttaacctgaacatgctggaacatctgaatcagcgctttgcgggcaactttgatacgcagcagtttgaacattgggcgttttataacgaacagctgaatcagattgaaatgcatctgcgctgtctggaagcgcataccgtgagactggaagcgctggatctgaccctgaactttgcggcgggcgaaaccattatgaccgaaattagccgcaaatttgatctggaaggcattcagcaagaactgcaagtgaaaggcctgaaaccgctgaaagtgtggaccgatagtcagcagtggtttggcctgattctgtgtcagcgctaa

SEQ ID NO: 3 - DNA, 40 bp, synthetic construct
  source 1..40: mol_type = other DNA
tttatgatgatcgcggctttgtgctgtttgaagaaatttg

SEQ ID NO: 4 - DNA, 40 bp, synthetic construct
  source 1..40: mol_type = other DNA
caaatttcttcaaacagcacaaagccgcgatcatcataaa

SEQ ID NO: 5 - DNA, 40 bp, synthetic construct
  source 1..40: mol_type = other DNA
tttatgatgatcgcggcctggtgctgtttgaagaaatttg

SEQ ID NO: 6 - DNA, 40 bp, synthetic construct
  source 1..40: mol_type = other DNA
caaatttcttcaaacagcaccaggccgcgatcatcataaa

SEQ ID NO: 7 - DNA, 40 bp, synthetic construct
  source 1..40: mol_type = other DNA
tttatgatgatcgcggcattgtgctgtttgaagaaatttg

SEQ ID NO: 8 - DNA, 40 bp, synthetic construct
  source 1..40: mol_type = other DNA
caaatttcttcaaacagcacaatgccgcgatcatcataaa

SEQ ID NO: 9 - DNA, 40 bp, synthetic construct
  source 1..40: mol_type = other DNA
tttatgatgatcgcggcatggtgctgtttgaagaaatttg

SEQ ID NO: 10 - DNA, 40 bp, synthetic construct
  source 1..40: mol_type = other DNA
caaatttcttcaaacagcaccatgccgcgatcatcataaa

SEQ ID NO: 11 - DNA, 40 bp, synthetic construct
  source 1..40: mol_type = other DNA
tttatgatgatcgcggcgtggtgctgtttgaagaaatttg

SEQ ID NO: 12 - DNA, 40 bp, synthetic construct
  source 1..40: mol_type = other DNA
caaatttcttcaaacagcaccacgccgcgatcatcataaa

SEQ ID NO: 13 - DNA, 40 bp, synthetic construct
  source 1..40: mol_type = other DNA
tttatgatgatcgcggcccggtgctgtttgaagaaatttg

SEQ ID NO: 14 - DNA, 40 bp, synthetic construct
  source 1..40: mol_type = other DNA
caaatttcttcaaacagcaccgggccgcgatcatcataaa

SEQ ID NO: 15 - DNA, 40 bp, synthetic construct
  source 1..40: mol_type = other DNA
tttatgatgatcgcggcaccgtgctgtttgaagaaatttg

SEQ ID NO: 16 - DNA, 40 bp, synthetic construct
  source 1..40: mol_type = other DNA
caaatttcttcaaacagcacggtgccgcgatcatcataaa

SEQ ID NO: 17 - DNA, 40 bp, synthetic construct
  source 1..40: mol_type = other DNA
tttatgatgatcgcggctatgtgctgtttgaagaaatttg

SEQ ID NO: 18 - DNA, 40 bp, synthetic construct
  source 1..40: mol_type = other DNA
caaatttcttcaaacagcacatagccgcgatcatcataaa

SEQ ID NO: 19 - DNA, 40 bp, synthetic construct
  source 1..40: mol_type = other DNA
tttatgatgatcgcggccatgtgctgtttgaagaaatttg

SEQ ID NO: 20 - DNA, 40 bp, synthetic construct
  source 1..40: mol_type = other DNA
caaatttcttcaaacagcacatggccgcgatcatcataaa

SEQ ID NO: 21 - DNA, 40 bp, synthetic construct
  source 1..40: mol_type = other DNA
tttatgatgatcgcggccaggtgctgtttgaagaaatttg

SEQ ID NO: 22 - DNA, 40 bp, synthetic construct
  source 1..40: mol_type = other DNA
caaatttcttcaaacagcacctggccgcgatcatcataaa

SEQ ID NO: 23 - DNA, 40 bp, synthetic construct
  source 1..40: mol_type = other DNA
tttatgatgatcgcggcaacgtgctgtttgaagaaatttg

SEQ ID NO: 24 - DNA, 40 bp, synthetic construct
  source 1..40: mol_type = other DNA
caaatttcttcaaacagcacgttgccgcgatcatcataaa

SEQ ID NO: 25 - DNA, 40 bp, synthetic construct
  source 1..40: mol_type = other DNA
tttatgatgatcgcggcaaagtgctgtttgaagaaatttg

SEQ ID NO: 26 - DNA, 40 bp, synthetic construct
  source 1..40: mol_type = other DNA
caaatttcttcaaacagcactttgccgcgatcatcataaa

SEQ ID NO: 27 - DNA, 40 bp, synthetic construct
  source 1..40: mol_type = other DNA
tttatgatgatcgcggcgatgtgctgtttgaagaaatttg

SEQ ID NO: 28 - DNA, 40 bp, synthetic construct
  source 1..40: mol_type = other DNA
caaatttcttcaaacagcacatcgccgcgatcatcataaa

SEQ ID NO: 29 - DNA, 40 bp, synthetic construct
  source 1..40: mol_type = other DNA
tttatgatgatcgcggcgaagtgctgtttgaagaaatttg

SEQ ID NO: 30 - DNA, 40 bp, synthetic construct
  source 1..40: mol_type = other DNA
caaatttcttcaaacagcacttcgccgcgatcatcataaa

SEQ ID NO: 31 - DNA, 40 bp, synthetic construct
  source 1..40: mol_type = other DNA
tttatgatgatcgcggctgcgtgctgtttgaagaaatttg

SEQ ID NO: 32 - DNA, 40 bp, synthetic construct
  source 1..40: mol_type = other DNA
caaatttcttcaaacagcacgcagccgcgatcatcataaa

SEQ ID NO: 33 - DNA, 40 bp, synthetic construct
  source 1..40: mol_type = other DNA
tttatgatgatcgcggctgggtgctgtttgaagaaatttg

SEQ ID NO: 34 - DNA, 40 bp, synthetic construct
  source 1..40: mol_type = other DNA
caaatttcttcaaacagcacccagccgcgatcatcataaa

SEQ ID NO: 35 - DNA, 40 bp, synthetic construct
  source 1..40: mol_type = other DNA
tttatgatgatcgcggccgcgtgctgtttgaagaaatttg

SEQ ID NO: 36 - DNA, 40 bp, synthetic construct
  source 1..40: mol_type = other DNA
caaatttcttcaaacagcacgcggccgcgatcatcataaa

SEQ ID NO: 37 - DNA, 40 bp, synthetic construct
  source 1..40: mol_type = other DNA
tttatgatgatcgcggcggcgtgctgtttgaagaaatttg

SEQ ID NO: 38 - DNA, 40 bp, synthetic construct
  source 1..40: mol_type = other DNA
caaatttcttcaaacagcacgccgccgcgatcatcataaa

(19) United States
(12) Patent Application Publication
(10) Pub. No.: US 2026/0110006 A1
(43) Pub. Date: Apr. 23, 2026

(54) HISTIDINE TRANSMETHYLASE MUTANT AND USE THEREOF

(71) Applicant: Tianjin University of Science and Technology, Tianjin (CN)

(72) Inventors: Heyun Wu, Tianjin (CN); Fan Li, Tianjin (CN); Xixian Xie, Tianjin (CN); Qian Ma, Tianjin (CN)

(21) Appl. No.: 19/078,320

(22) Filed: Mar. 13, 2025

(30) Foreign Application Priority Data
Oct. 18, 2024 (CN) 202411463132.8

(51) Int. Cl. C12P 17/10; C12N 9/10; C12N 15/70

(52) U.S. Cl. CPC C12P 17/10; C12N 9/1007; C12N 15/70; C12Y 201/01044

(57) ABSTRACT

The present invention falls within the technical field of genetic engineering, and specifically provides a histidine transmethylase mutant and use thereof. The mutant protein has significantly improved catalytic activity over a parent protein and thus, promotes the production of ergothioneine (L-EGT) catalyzed by the histidine transmethylase, which provides a novel synthetic pathway and engineering bacterium for the production of L-EGT.

### CROSS REFERENCE TO RELATED APPLICATION

This application claims priority of Chinese Application No. 202411463132.8 filed on Oct. 18, 2024 and entitled “Histidine transmethylase mutant and use thereof”, the contents of which are incorporated herein by reference in their entirety.

### REFERENCE TO SEQUENCE LISTING

This application includes a Sequence Listing filed electronically as an XML file named “Sequence listing_RSMK-25001-USPT.xml”, created on Mar. 5, 2025, with a size of 36,601 bytes. The Sequence Listing is incorporated herein by reference.

### TECHNICAL FIELD

The present invention falls within the technical field of genetic engineering, and specifically relates to a histidine transmethylase mutant and use thereof.

### BACKGROUND

Ergothioneine (EGT for short), also called a thiolhistidine trimethyl inner salt, with a molecular formula of C₉H₁₅N₃O₂S, was discovered in Ergothioneine E. coli in 1909 for the first time and gets its name. EGT is a kind of natural antioxidant and can scavenge intracellular active radicals to inhibit the activity of certain oxidases and lots of intracellular oxidation reactions. Therefore, EGT has been widely applied in the fields such as food, medicine, cosmetics, health-care products, and biotechnology. At present, EGT can be synthesized by chemical approaches, and also can be extracted from mushrooms and other natural products. However, there are the shortcomings of low output, more impurities, and high costs in the two methods. EGT is produced via microbiological fermentation process; the process is low in raw material costs, eco-friendly, easy to operate, short in period, and thus suitable for industrial production; hence, the process has become a hot spot in the production and research of the existing EGT.

EGT-producing microorganisms have low yield and long growth cycle and thus, cannot meet the requirements of industrial production. The first step of the biosynthetic pathway of EGT is that histidine is catalyzed by a SAM (S-adenosylmethionine)-dependent histidine trimethylase (EgtD) and transformed into Hercynine (HER for short). The step plays an important role in the synthesis of EGT, and EGT cannot be synthesized in case of no the step. Vit, et al., represented EgtD in 2015; the turn over number of the enzyme to histidine was only 0.58 s⁻¹ (Misson, Laetitia, Chembiochem: A European journal of chemical biology, 2015.), which was 2.3% activity of the most active EgtE enzyme in the metabolic pathway. Therefore, EgtD is a key rate-limiting step to the synthesis of EGT. Currently, studies have reported the crystal structure of EgtD (for example, PDB: 4UY5, 4UY7, and 4UY6; JEONG J H, CHA H J, HAS C, et al. Structural insights into the histidine trimethylation activity of EgtD from Mycobacterium smegmatis[J]. Biochem Biophys Res Commun, 2014, 452(4):1098-1103), and related enzymatic properties have been studied. It has been found through the existing studies that catalyzing histidine for trimethylation via EgtD is a continuous process, and its catalytic activity is regulated and controlled by a strict substrate. Therefore, there is an urgent need for a high-enzymatic activity EgtD to solve the problem, i.e., transmethylase has a low enzyme activity and thus, does not comply with the requirements of industrial production.

### SUMMARY

Directed to the shortcomings in the prior art, the objective of the present invention is to provide a histidine transmethylase (EgtD) mutant and use thereof. The mutant protein has significantly improved catalytic activity relative to a parent protein and thus, promotes the production of EGT catalyzed by EgtD.

The technical solution of the present invention is as follows:

The present invention provides a histidine transmethylase EgtD mutant, whose substitution relative to a parent histidine transmethylase is selected from one of the following: S70V, S70N, S70K, and S70W; and each position is corresponding to an amino acid sequence of the parent histidine transmethylase as shown in SEQ ID NO: 1.

The present invention further provides an encoding gene of the aforesaid EgtD mutant.

The present invention further provides a vector, a recombinant vector, or an expression vector containing the aforesaid encoding gene.

The present invention further provides a host cell containing the aforesaid encoding gene or recombinant vector.

The present invention further provides use of the aforesaid EgtD mutant, where the mutant protein is for use in catalyzing histidine to be transformed into hercynine (HER).

The present invention further provides a method for preparing the aforesaid EgtD mutant, including performing genetic recombination and expression by utilizing the encoding gene of the EgtD mutant or an expression vector including the encoding gene.

The present invention further provides a method for producing ergothioneine (L-EGT), including a step of introducing the EgtD mutant into a microorganism expression system including EgtA, EgtB, EgtC, and EgtE, and preparing L-EGT with L-His and L-Met as substrates under appropriate conditions.

The present invention further provides a method for improving enzyme activity of histidine transmethylase, comprising introducing the following substitution: S70N, S70K, S70K, or S70W into a parent histidine transmethylase; wherein each position is corresponding to an amino acid sequence of the parent histidine transmethylase as shown in SEQ ID NO: 1.

Advantageous effects of the present invention are as follows:

1. The EgtD protein is transformed by means of directed evolution of active sites and gene mutation, and screened to obtain a mutant with improved activity.

2. The mutant with improved activity is combined with other enzymes (e.g., EgtA, EgtB, EgtC, and EgtE) in the synthetic pathway of EGT for the biosynthesis of EGT, thus improving the microbial fermentation level of EGT.

3. The present invention obtains the key amino acid site with improved EgtD enzyme activity for the first time, being the active site at position 70 of the EgtD enzyme derived from chlorella; the above key amino acid site can be mutated to effectively improve the EgtD enzyme activity, thus providing a key target for the further improvement of its subsequent enzyme activity.

4. An Escherichia coli expression system is set as an example in the present invention to obtain an engineering bacterium containing an EgtD mutant, and the engineering bacterium is applied in the production of EGT. The mutant is found to effectively improve the output of EGT. Compared to non-mutated engineering bacteria, the engineering bacterium containing the mutant improves the output of EGT by 22-24 times. All the mutants in the present invention effectively improve the synthetic efficiency of EGT, which provides a novel synthetic pathway and engineering bacterium for the production of EGT.

### BRIEF DESCRIPTION OF THE DRAWINGS

FIG. 1 shows an amino acid sequence of a parent histidine transmethylase according to the present invention;

FIG. 2 shows a nucleotide sequence of an encoding gene of the parent histidine transmethylase according to the present invention;

FIG. 3 shows a profile of a plasmid expressing the histidine transmethylase (EgtD) in an example;

FIG. 4 shows a test result of flask shaking fermentation of an alanine-induced strain in an example; and

FIG. 5 shows a test result of flask shaking fermentation of an S70 saturated mutant strain in an example.

### DETAILED DESCRIPTION OF EMBODIMENTS

The present invention will be set forth with detailed embodiments below. Unless otherwise specified particularly, technical means used in the present invention are all conventional methods for those skilled in the art. In addition, the embodiments should be understood as being illustrative instead of limiting the scope of the present invention. The essence and scope of the present invention are merely defined by the claims. In the premise of not departing from the essence and scope of the present invention, various changes or alterations to the material components and use amount in these embodiments also fall within the scope of protection of the present invention to those skilled in the art.

The present invention provides a histidine transmethylase EgtD mutant, whose substitution relative to a parent histidine transmethylase is selected from one of the following: S70V, S70N, S70K, and S70W; and each position is corresponding to an amino acid sequence of the parent histidine transmethylase as shown in SEQ ID NO: 1.

The present invention further provides an encoding gene of the aforesaid EgtD mutant.

The present invention further provides a vector, a recombinant vector, or an expression vector containing the aforesaid encoding gene; for example, the recombinant vector constituted by a plasmid pTrc99a and the encoding gene of the present invention.

The present invention further provides a host cell containing the aforesaid encoding gene or recombinant vector. The host cell may be any host suitable for generating the EgtD mutant of the present invention from the gene or vector in the present invention; for example, Escherichia coli, Bacillus subtilis, Saccharomyces cerevisiae, and other microorganism expression systems.

The present invention further provides use of the aforesaid EgtD mutant, where the mutant protein is for use in catalyzing histidine to be transformed into hercynine (HER).

The aforesaid EgtD mutant of the present invention has improved enzyme activity as compared to its parent EgtD; for example, in some embodiments, enzyme activity is improved by 5 times above; in some other embodiments, enzyme activity is improved by 10 above; in a further embodiment, enzyme activity is improved by 20 times above; and in a further embodiment, enzyme activity is improved by 30 times above.

The present invention further provides a method for preparing the aforesaid EgtD mutant, including performing genetic recombination and expression by utilizing the encoding gene of the EgtD mutant or an expression vector including the encoding gene. Genetic recombination methods and expression hosts known to those skilled in the art may be used, and media and culture conditions suitable for expression of hosts are selected. The method may further include a step of recycling the EgtD mutant; the recovery step may relate to a step of isolating or purifying the EgtD mutant from a culture or expression product of the host; any method known to those skilled in the art may be used.

The present invention further provides a method for producing ergothioneine (L-EGT), including a step of introducing the EgtD mutant into a microorganism expression system including EgtA, EgtB, EgtC, and EgtE (e.g., Escherichia coli expression system), and preparing L-EGT with L-His and L-Met as substrates under appropriate conditions.

In the aforesaid method of producing EGT, preferably, the medium consists of: 20-40 g/L glucose, 1-4 g/L yeast extract, 2-5 g/L peptone, 1-2 g/L sodium citrate, 1-2 g/L KH₂PO₄, 1-2 g/L MgSO₄·7H₂O, 10-20 mg/L FeSO₄, 1-2 mg/L V_{B·mix}(1,3,5,12), 1-2 mg/L V_{H}, 1-10 mg/L V_{B6}, 1-10 mg/L MnSO₄, water as a remainder, pH: 7.0-7.2.

The present invention further provides a method for improving enzyme activity of histidine transmethylase, comprising introducing the following substitution: S70N, S70K, S70K, or S70W into a parent histidine transmethylase; wherein each position is corresponding to an amino acid sequence of the parent histidine transmethylase as shown in SEQ ID NO: 1.

The following definitions are adopted in the present invention:

### 1. Terminology of Amino Acids and DNA Nucleotide Sequences

A universally acknowledged IUPAC terminology is used for amino acid residues in a form of three-letter abbreviation or a single alphabetic symbol. A universally acknowledged IUPAC terminology is adopted for DNA nucleotide sequences.

### 2. Identification of EgtD Mutants

The “amino acid replaced by the original position of amino acid” denotes the mutational amino acid in the EgtD mutant. For example, S70K denotes that the amino acid residue in position 70 of the amino acid sequence starting from the N terminal to the C terminal in sequence is replaced by lysine (K) from the serine(S) of the parent EgtD. The numbering way of the EgtD amino acid in the present invention is based on the sequence as shown in SEQ ID NO: 1.

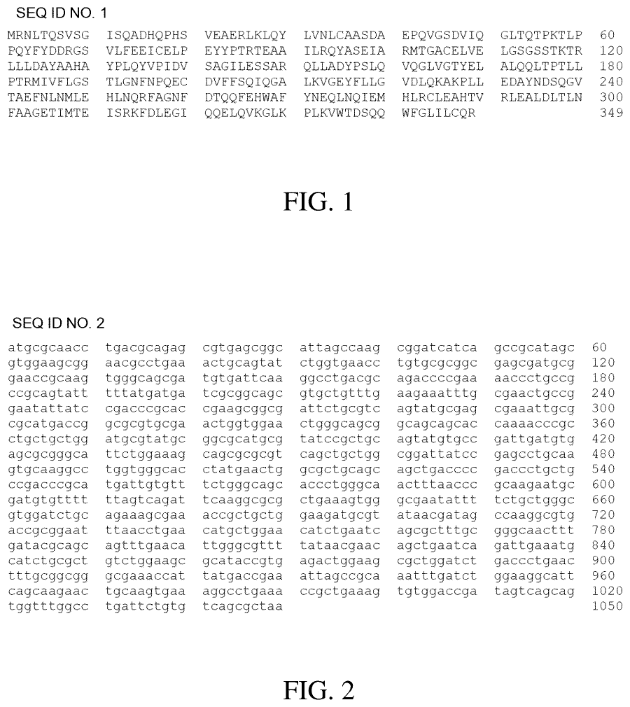

3. The amino acid sequence (SEQ ID NO: 1) of the parent histidine transmethylase EgtD is shown in FIG. 1; the encoding gene sequence (SEQ ID NO: 2) is shown in FIG. 2.

The present invention will be described more specifically below through detailed examples. Unless otherwise defined, the technical and scientific terms used in the following examples have the same meaning as commonly understood by those skilled in art of the present invention.

### Example 1. Preparation Method of the Mutant

### 1.1 Primer Design

A primer design software primer5 was used and EgtD enzyme sequence served as a template; primers were designed at the head and the tail of a gene of interest; the EgtD enzyme sequence served as a template, and amplification primers containing mutation site bases were designed at the mutation site. Forward and reverse primers were amplified by a PCR method, and recombinant fragments were prepared by overlapping PCR.

PCR amplification system is as follows:

| Component | Volume (50 μL) | |
|---|---|---|
| DNA template | 1 | μL |
| Forward primer (10 μmol/L) | 1 | μL |
| Reverse primer (10 μmol/L) | 1 | μL |
| dNTP mixture (10 mmol/L) | 4 | μL |
| 5 × Buffer | 10 | μL |
| HS enzyme (5 U/μL) | 0.5 | μL |
| ddH₂O | 32.5 | μL |

Overlap PCR system is as follows:

| | | |
|---|---|---|
| Template | 2 | μL |
| Forward primer of the forward homologous | 1 | μL |
| arm (10 μmol/L) | | |
| Reverse primer of the reverse homologous arm | 1 | μL |
| (10 μmol/L) | | |
| dNTP mixture (10 mmol/L) | 4 | μL |
| 5 × Buffer | 10 | μL |
| HS enzyme (5 U/μL) | 0.5 | μL |
| ddH₂O | 31.5 | μL |

PCR reaction conditions (PrimeSTAR HS enzyme, Takara): pre-denatured for 5 min at 95° C.; 30 rounds of cycles were performed, denatured for 10 s at 98° C., annealed for 15 s at (Tm-3/5)° C., extended at 72° C. (extended to about 1 kb at the enzyme activity for 1 min), and then continuously extended at 72° C. for 10 min, and maintained at 4° C.

1.2 Preparation of a liner vector: a vector was linearized by inverse PCR amplification.

1.3 Plasmid construction method: target fragments were obtained by PCR, and the plasmid was subjected to inverse PCR amplification, and DNA gel extraction was performed, respectively, to obtain fragments of interest and vector fragments, then homologously recombined, and transformed into Escherichia coli DH5α competent cells, and coated on a resistant plate. A single colony identified as positive was picked up and cultured to extract the plasmid.

1.4 Recombination reaction: the used recombinases were all the enzymes of series ClonExpress® II One Step Cloning Kit; and recombination condition: for 30 min at 37° C.

Recombination system is as follows:

| Reaction system | Volume (20 μL) |
|---|---|
| 5 × CE II Buffer | 4 μL |
| Linearized cloning vector | 1 μL |
| Fragment-inserted cloning vector | 1 μL |
| Exnase ® II | 2 μL |
| ddH₂O | 12 μL |

1.5 Plasmid transformation: 10 μL reaction fluid was taken, added and transformed to 100 mL of DH5α competent cells, slightly mixed well, and put into an ice bath for 20 min, and subjected to heat shock for 45-90 s at 42° C., and immediately put into an ice bath for 2-3 min, added with 900 μL of SOC, and resuscitated for 1 h at 37° C. The cells were centrifuged for 2 min at 8000 rpm, and partial supernatant was discarded, and about 200 μL of the supernatant was retained; bacterial cells were resuspended and coated onto a plate containing 100 mg/L ampicillin, and then the plate was inverted and cultured over the night at 37° C. Single colonies were grown on the plate and positive recombinants were picked up by PCR identification of the colonies.

1.6 Clone identification: PCR positive colonies were inoculated into an LB medium containing 100 mg/L of ampicillin and cultured over the night, bacteria were retained to extract the plasmid, and identification was performed by restriction analysis.

TABLE 1
Primers for PCR amplification (S70 site saturated mutation)

| | | SEQ | |
|---|---|---|---|
| Primer | | ID | Mutation |
| name | Nucleotide sequence | NO | site |
| D1-S | TTTATGATGATCGCGGCTTTGTGCTGTTTGAAGAAATTTG | 3 | S70F |
| D1-A | CAAATTTCTTCAAACAGCACAAAGCCGCGATCATCATAAA | 4 | |
| D2-S | TTTATGATGATCGCGGCCTGGTGCTGTTTGAAGAAATTTG | 5 | S70L |
| D2-A | CAAATTTCTTCAAACAGCACCAGGCCGCGATCATCATAAA | 6 | |
| D3-S | TTTATGATGATCGCGGCATTGTGCTGTTTGAAGAAATTTG | 7 | S70I |
| D3-A | CAAATTTCTTCAAACAGCACAATGCCGCGATCATCATAAA | 8 | |
| D4-S | TTTATGATGATCGCGGCATGGTGCTGTTTGAAGAAATTTG | 9 | S70M |
| D4-A | CAAATTTCTTCAAACAGCACCATGCCGCGATCATCATAAA | 10 | |
| D5-S | TTTATGATGATCGCGGCGTGGTGCTGTTTGAAGAAATTTG | 11 | S70V |
| D5-A | CAAATTTCTTCAAACAGCACCACGCCGCGATCATCATAAA | 12 | |
| D6-S | TTTATGATGATCGCGGCCCGGTGCTGTTTGAAGAAATTTG | 13 | S70P |
| D6-A | CAAATTTCTTCAAACAGCACCGGGCCGCGATCATCATAAA | 14 | |
| D7-S | TTTATGATGATCGCGGCACCGTGCTGTTTGAAGAAATTTG | 15 | S70T |
| D7-A | CAAATTTCTTCAAACAGCACGGTGCCGCGATCATCATAAA | 16 | |
| D8-S | TTTATGATGATCGCGGCTATGTGCTGTTTGAAGAAATTTG | 17 | S70Y |
| D8-A | CAAATTTCTTCAAACAGCACATAGCCGCGATCATCATAAA | 18 | |
| D9-S | TTTATGATGATCGCGGCCATGTGCTGTTTGAAGAAATTTG | 19 | S70H |
| D9-A | CAAATTTCTTCAAACAGCACATGGCCGCGATCATCATAAA | 20 | |
| D10-S | TTTATGATGATCGCGGCCAGGTGCTGTTTGAAGAAATTTG | 21 | S70Q |
| D10- | CAAATTTCTTCAAACAGCACCTGGCCGCGATCATCATAAA | 22 | |
| A | | | |
| D11-S | TTTATGATGATCGCGGCAACGTGCTGTTTGAAGAAATTTG | 23 | S70N |
| D11- | CAAATTTCTTCAAACAGCACGTTGCCGCGATCATCATAAA | 24 | |
| A | | | |
| D12-S | TTTATGATGATCGCGGCAAAGTGCTGTTTGAAGAAATTTG | 25 | S70K |
| D12- | CAAATTTCTTCAAACAGCACTTTGCCGCGATCATCATAAA | 26 | |
| A | | | |
| D13-S | TTTATGATGATCGCGGCGATGTGCTGTTTGAAGAAATTTG | 27 | S70D |
| D13- | CAAATTTCTTCAAACAGCACATCGCCGCGATCATCATAAA | 28 | |
| A | | | |
| D14-S | TTTATGATGATCGCGGCGAAGTGCTGTTTGAAGAAATTTG | 29 | S70E |
| D14- | CAAATTTCTTCAAACAGCACTTCGCCGCGATCATCATAAA | 30 | |
| A | | | |
| D15-S | TTTATGATGATCGCGGCTGCGTGCTGTTTGAAGAAATTTG | 31 | S70C |
| D15- | CAAATTTCTTCAAACAGCACGCAGCCGCGATCATCATAAA | 32 | |
| A | | | |
| D16-S | TTTATGATGATCGCGGCTGGGTGCTGTTTGAAGAAATTTG | 33 | S70W |
| D16- | CAAATTTCTTCAAACAGCACCCAGCCGCGATCATCATAAA | 34 | |
| A | | | |
| D17-S | TTTATGATGATCGCGGCCGCGTGCTGTTTGAAGAAATTTG | 35 | S70R |
| D17- | CAAATTTCTTCAAACAGCACGCGGCCGCGATCATCATAAA | 36 | |
| A | | | |
| D18-S | TTTATGATGATCGCGGCGGCGTGCTGTTTGAAGAAATTTG | 37 | S70G |
| D18- | CAAATTTCTTCAAACAGCACGCCGCCGCGATCATCATAAA | 38 | |
| A | | | |

### Example 2. Construction and Fermentation Test of the Mutant Strain

### 2.1 Construction of the Alanine-Induced Strain

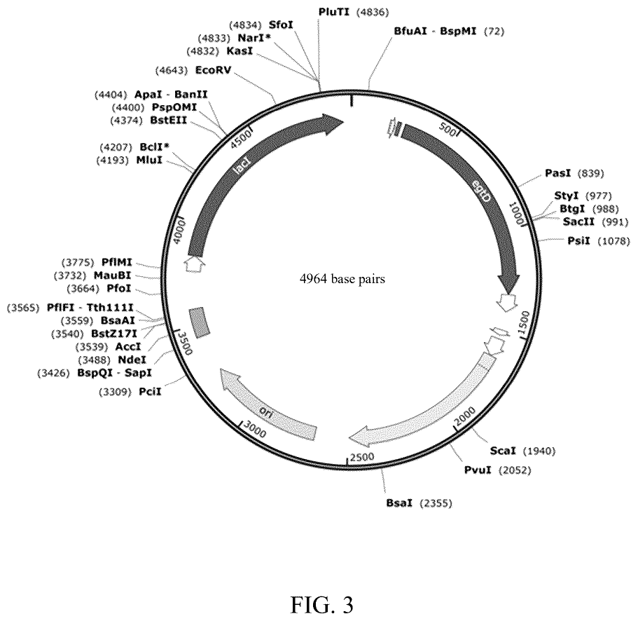

The EgtD enzyme, a substrate SAM and L-histidine were subjected to molecular docking by Discovery studio software, respectively to simulate their potential interactions. Amino acid residue sites were obtained according to the molecular docking results (Phe73, Ser115, Glu74, Ser70, Cys77, Lys118, Asp139, Tyr65, Ser312, Met280, Glu310, Gly193, Phe64, Tyr234, Phe244, Ile311, and Asn245). By primer designing, the amino acids at the corresponding site were replaced with alanine, to obtain mutant genes egtD^{F73A}, egtD^{S115A}, egtD^{E74A}, egD^{S70A}, egtD^{C77A}, egtD^{K118A}, egtD^{P139A}, egtD^{Y65A}, egtD^{S312A}, egtD^{M280A}, egtD^{E310A}, egtD^{G193A}, egtD^{F64A}, egtD^{Y234A}, egtD^{F244A}, egtD^{I311A}, and egtD^{N245A}. Afterwards, the mutant fragment was ligated to a linearized vector pTrc99a (FIG. 3) to obtain a recombinant plasmid. The recombinant plasmid was transformed into DH5α competent cells, after spread plate cultivation, several clones were picked out from the recombination transformation plate for PCR identification of bacterial colonies. The bacterial colonies identified as positive by PCR were cultured and a plasmid was extracted. Afterwards, the plasmid was transformed into an EGT-producing strain to obtain an alanine scanning mutant strain.

### 2.2 Key Site Selection for Site Directed Mutagenesis

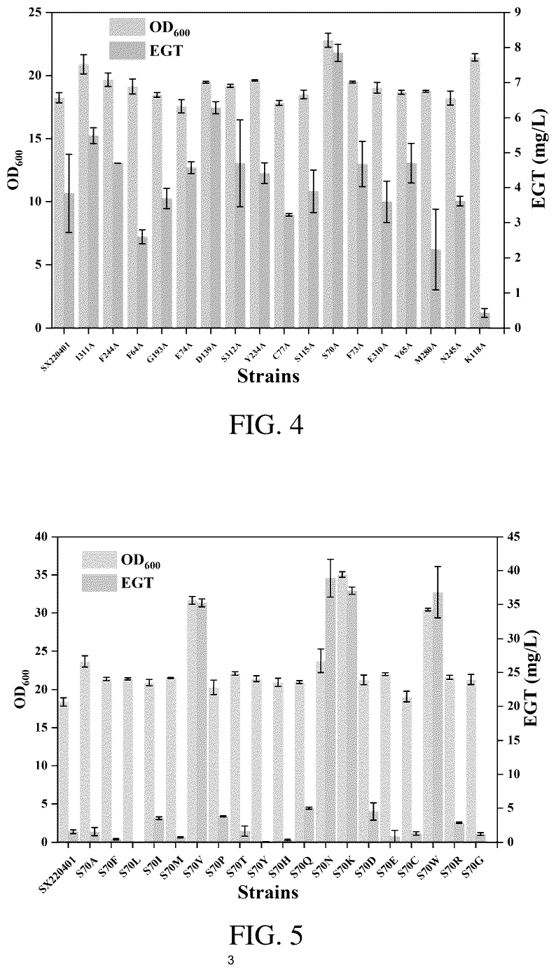

10 μL of glycerol stock (a mutant strain) was taken and inoculated into 5 mL of an LB, and cultured for 12 h at 37° C. and 220 rpm. A seed solution was transferred into a shake flask containing 30 mL of LB medium in an inoculum size of 3%, and then cultured for 12 h at 37° C. and 220 rpm. A seed solution was transferred into a fermentation flask containing 30 mL of LB medium in an inoculum size of 10%, and then cultured for 24 h at 37° C. and 220 rpm. At the end of fermentation, the fermentation liquor was treated by a fermentation liquor treatment method and detected by high-performance liquid chromatography. Fermentation results are shown in FIG. 4 and Table 2. In all the mutants, the S70A mutant has the most significantly improved output of EGT as compared to the control strain by 105% and thus, the S70A is determined as the key site for the binding of EgtD to a substrate.

TABLE 2
Result of flask shaking fermentation of the alanine-induced strain

| Mutation site | OD₆₀₀ | EGT (mg/L) |
|---|---|---|
| SX220401 | 18.2 | 3.83 |
| I311A | 20.8 | 5.48 |
| F244A | 19.6 | 4.70 |
| F64A | 19.1 | 2.59 |
| G193A | 18.4 | 3.68 |
| E74A | 17.5 | 4.57 |
| D139A | 19.4 | 6.28 |
| S312A | 19.1 | 4.69 |
| Y234A | 19.6 | 4.41 |
| C77A | 17.8 | 3.22 |
| S115A | 18.5 | 3.89 |
| S70A | 22.8 | 7.84 |
| F73A | 19.4 | 4.6 |
| E310A | 19.3 | 3.59 |
| Y65A | 18.6 | 4.69 |
| M280A | 18.7 | 2.23 |
| N245A | 18.2 | 3.61 |
| K118A | 21.4 | 0.43 |

### 2.3 Construction of a Saturated Mutant Strain

By primer designing (Table 1), serine at position 70 was mutated into the rest 18 amino acids to obtain the mutant genes egtD^{S70F}, egtD^{S70L}, egtD^{S70I}, egtD^{S70M}, egtD^{S70V}, egtD^{S70W}, egtD^{S70P}, egtD^{S70T}, egtD^{S70Y}, egtD^{S70H}, egtD^{S70Q}, egtD^{S70N}, egtD^{S70K}, egtD^{S70C}, egtD^{S70R}, egtD^{S70G}, egtD^{S70D}, egtD^{S70E}. Afterwards, the mutant fragment was ligated to a linearized vector pTrc99a to obtain a recombinant plasmid. The recombinant plasmid was transformed into DH5α competent cells, after spread plate cultivation, several clones were picked out from the recombination transformation plate for PCR identification of bacterial colonies. The bacterial colonies identified as positive by PCR were cultured and a plasmid was extracted. Afterwards, the plasmid was transformed into an EGT-producing strain SX180110(MG1655ΔlacIZ::P_{xylF}-T7RNAP,Δmlc::mlc*,ΔpurR,ΔtdcD::P_{trc}-hisG,ΔyghX::P_{trc}-hisDCBHAFI,ΔilvG::Ptrc-egtBCDE,ΔmbhA::P_{trc}-gshA,ΔtehB::P_{trc}-egtE_{ncr}) to obtain a saturated mutant strain.

2.4 Obtaining of the Mutants with Improved Catalytic Activity and its EGT-Producing Strain

The saturated mutant strain and the control strain SX220401(SX180110/pTrc99a-egtDₘᵥₐ) were subjected to flask shaking fermentation test for detection. The fermentation results are shown in FIG. 5 and Table 3. The production capacity of EGT in mutant strains such as S70N, S70K, S70W, and S70V is improved; the EGT output is improved by 24 folds, 23 folds, 23 folds, and 22 folds, respectively, as compared to the control strain. These results indicate that the four mutant enzymes achieve higher catalytic activity as compared to the parent enzyme.

TABLE 3
Results of flask shaking fermentation
of the S70 saturated mutant strain

| Mutation site | OD₆₀₀ | EGT (mg/L) |
|---|---|---|
| SX220401 | 18.3 | 1.53 |
| S70G | 21.3 | 1.21 |
| S70F | 21.3 | 0.45 |
| S70L | 21.4 | 0.07 |
| S70I | 20.9 | 3.51 |
| S70M | 21.5 | 0.72 |
| S70V | 31.6 | 35.22 |
| S70P | 20.3 | 3.77 |
| S70T | 22.8 | 1.63 |
| S70Y | 21.4 | 0.08 |
| S70H | 20.9 | 0.33 |
| S70Q | 20.9 | 4.98 |
| S70N | 23.7 | 38.89 |
| S70K | 35. | 37.06 |
| S70D | 21.2 | 4.50 |
| S70E | 22.3 | 0.84 |
| S70C | 19.6 | 1.28 |
| S70W | 30.4 | 36.80 |
| S70R | 21.6 | 2.86 |

Flask shaking fermentation: bacterial cells were activated to prepare a seed solution; the seed solution was inoculated into a 500 mL of conical flask (final volume of 30 mL) in an inoculum size of 10%, and the conical flask was sealed with 9 layers of gauzes for shaking culture at 37° C. and 200 r/min; ammonia water was supplemented during the fermentation to keep a pH value of 7.0; and 60% (m/v) of glucose solution was supplemented to maintain the fermentation.

Preferably, the medium composition is as follows: 20 g/L glucose, 4 g/L yeast extract, 5 g/L peptone, 2 g/L sodium citrate, 2 g/L KH₂PO₄, 2 g/L MgSO₄·7H₂O, 20 mg/L FeSO₄, 2 mg/L V_{B·mix}(1,3,5,12), 2 mg/L V_{H}, 10 mg/L V_{B6}, 10 mg/L MnSO₄, water as a remainder, pH: 7.0-7.2.

Treatment method of the fermentation liquor: 1 mL of the fermentation liquor was taken and centrifuged for 3 min at 13000 rpm; supernatant was taken and filtered with 0.22 μm of a filter membrane, and then subjected to HPLC analysis.

HPLC conditions: 2% (v/v) acetonitrile as a mobile phase, flow rate of 0.7 mL/min, a chromatographic column of a Titank C18 column (250 mm×4.60 mm, 5 μm), column temperature of 30° C., ultraviolet detection wavelength of 257 nm, and injection volume of 10 μL.

The present invention has been disclosed by the above preferred embodiments, but is not construed as limiting the present invention. Those skilled in the art can make various forms and details of changes, amendments, replacements, and transformations on these embodiments without departing from the spirit and principle of the present invention. The scope of the present invention is defined here by the claims and equivalents, and typed into the disclosure of the description.

What is claimed is:

1. A histidine transmethylase EgtD mutant, wherein a substitution of the EgtD mutant relative to a parent histidine transmethylase is selected from one of the following: S70V, S70N, S70K, and S70W; and each position is corresponding to an amino acid sequence of the parent histidine transmethylase as shown in SEQ ID NO: 1.

2. An encoding gene of the EgtD mutant of claim 1.

3. A recombinant vector, comprising the encoding gene of claim 2.

4. A host cell, comprising the encoding gene of claim 2.

5. The host cell according to claim 4, wherein the host cell is Escherichia coli.

6. Use of the EgtD mutant according to claim 1, wherein the mutant protein is for use in catalyzing histidine to be transformed into hercynine (HER).

7. A method for preparing the EgtD mutant according to claim 1, comprising performing genetic recombination and expression by utilizing the encoding gene of the EgtD mutant or an expression vector comprising the encoding gene.

8. A method for producing ergothioneine (L-EGT), comprising a step of introducing the EgtD mutant of claim 1 into a microorganism expression system comprising EgtA, EgtB, EgtC, and EgtE, and preparing L-EGT with L-His and L-Met as substrates under appropriate conditions.

9. The method according to claim 8, wherein the microorganism expression system is Escherichia coli.

10. A method for improving enzyme activity of a histidine transmethylase, comprising introducing the following substitution: S70N, S70K, S70K, or S70W into a parent histidine transmethylase; wherein each position is corresponding to an amino acid sequence of the parent histidine transmethylase as shown in SEQ ID NO: 1.

11. A host cell, comprising the recombinant vector of claim 3.
